(19) United States
(12) Patent Application Publication
(10) Pub. No.: US 2026/0131097 A1
(43) Pub. Date: May 14, 2026

(54) NASAL CANNULA INCLUDING ADJUSTABLE BOLO CLASP

(71) Applicant: Ronald Martin, Denver, CO (US)

(72) Inventor: Ethan James Pappas, Columbus, OH (US)

(73) Assignee: Ronald Martin, Denver, CO (US)

(21) Appl. No.: 18/943,095

(22) Filed: Nov. 11, 2024

(51) Int. Cl. A61M 16/06

(52) U.S. Cl. CPC A61M 16/0672

(57) ABSTRACT

A nasal cannula includes a flexible tube fluidly connected to a pair of prongs to be received in nose nostrils. Two ends of the tube are received through a bolo clasp whereby the tube defines a loop closed by the clasp. In one embodiment, the bolo clasp includes a pair of jaw members which are pivotally secured together with elastic/spring hinges and define a space wherethrough the tube ends are received. The jaw members pivot between an open position whereat the clasp can slide along the tubes and a clamping position whereat the jaws engage the tubes and prevent sliding. In another embodiment, the bolo clasp includes a tube-shaped body having a receiving slot wherethrough the tube ends are received. The body and slot deform between an open position whereat the clasp/body can slide along the tubes and a clamping position whereat the body engages the tubes and prevents sliding.

### BACKGROUND OF THE INVENTION

### 1. Field of the Invention

The present invention relates to nasal cannulas. More particularly, the present invention is directed to nasal cannulas including a bolo clasp which is slidingly adjustable along the cannula flexible oxygen delivery tubes for fitting the cannula loop on the patient/person and which selectively engages/clamps onto the tubes for preventing inadvertent displacement of the loop and displacement/removal of the cannula prongs from the nostrils.

### 2. Background

Nasal cannulas are used for supplying supplemental oxygen to patients/persons who are having trouble breathing or for some other reason require supplemental oxygen and are typically used by fluidly connecting the cannula flexible tubes to an oxygen source, such as, for example, a portable oxygen generator or tank. The nasal cannula includes a pair of prongs which are fluidly connected to the flexible tubing, and which are adapted to be received in the patient's/person's nostrils. The flexible tubing forms a cannula loop which extends along and between the upper lip and the nose and is hooked over the patient's/person's ears and under the chin to thereby retain the prongs in the nostrils. The ends of the flexible tubing loop are received through a bolo clasp which closes the loop and which is slidingly adjustable along the tubing for increasing and decreasing the size of the loop and thereby adjusting the fitment of the cannula on the patient/person. Traditionally, the bolo clasp is formed from a flexible ring or band which cinches around the flexible tubing and is slidingly adjusted along the length of the tubing for increasing or decreasing the size of the loop.

Traditional bolo clasps, however, are known to slip along the flexible tubes during use which increases the size of and loosens the cannula loop and causes the cannula to be displaced or fall of the patient/person entirely and causes the prongs to exit the nostrils. Slippage of the bolo clasp and loosening of the cannula loop can also cause displacement of the prongs such as while a patient/person is sleeping and turns their head thereby forcing the cannula loop against a pillow. Accordingly, there exists a need for an improved nasal cannula including a bolo clasp which can be easily adjusted along the flexible tubes and which resists slipping along the flexible tubes during use.

### SUMMARY OF THE INVENTION

The nasal cannulas constructed in accordance with the principles of the present invention overcome disadvantages of prior traditional nasal cannulas. In this regard, in one for thereof, the present invention is directed a nasal cannula which includes a flexible tube fluidly connected to a pair of prongs adapted to be received in nose nostrils for delivering oxygen to the prongs; a bolo clasp comprising a pair of jaw members pivotally secured together with one or more hinges and defining one or more spaces therebetween; wherein two ends of the flexible tube are received through the bolo clasp one or more spaces whereby a cannula loop is defined by the flexible tube and is closed by the bolo clasp; and wherein the jaw members pivot about the one or more hinges between an open position whereat the bolo clasp can be slidingly adjusted along the flexible tubes for thereby adjusting the size of the loop and a clamping position whereat the jaw members engage the flexible tubes and prevent sliding of the bolo clasp along the flexible tubes.

Preferably, one or more of the hinges are spring hinges. For example: the jaw members and the one or more hinges can be integrally formed from one or more polymer materials and the one or more hinges can be live hinges; or the jaw members and the one or more hinges can be integrally formed from one or more polymer materials and the one or more hinges can be elastic live hinges having shape memory; or the jaw members and one or more hinges can be integrally formed from one or more polymer materials and the one or more hinges can be live hinges and, further, one or more metallic spring members can be embedded within each of the one or more live hinges; or the jaw members and the one or more hinges can be integrally formed from one or more polymer materials and the one or more hinges can be elastic live hinges having shape memory and, further, one or more metallic spring members can be embedded within each of the one or more live hinges; or the one or more hinges can include a metallic spring member.

More preferably each jaw member can include a lever tab and the one or more hinges can be located between the jaw members and lever tabs whereby the hinges also function as fulcrum points, and each jaw member can include an impingement area adapted to engage the flexible tubes and prevent sliding of the bolo clasp along the flexible tubes. Also, each jaw member can include a pair of grooves facing the one or more spaces between the jaws and aligned with the pair of grooves of the opposing jaw thereby defining a pair of tube receiving channels whereat the flexible tubes are received. The polymer material from which the clasp is made is one or more of polyurethane, polyvinyl chloride, silicon rubber and styrene-butadiene rubber.

In another form thereof, the present invention is directed to a nasal cannula including a flexible tube fluidly connected to a pair of prongs adapted to be received in nose nostrils for delivering oxygen to the prongs; a bolo clasp comprising a tube-shaped body having a tube receiving slot extending therethrough; wherein two ends of the flexible tube are received through the bolo clasp tube receiving slot whereby a cannula loop is defined by the flexible tube and is closed by the bolo clasp; and, wherein the tube-shaped body deforms between an open position whereat the bolo clasp can be slidingly adjusted along the flexible tubes for thereby adjusting the size of the loop and a clamping position whereat the tube-shaped body engages the flexible tubes and prevents sliding of the bolo clasp along the flexible tubes.

Preferably, the tube-shaped body is integrally formed from one or more polymer materials; or the tube-shaped body is integrally formed from one or more elastic polymer materials which has shape memory; or the tube-shaped body is integrally formed from one or more spring steel materials; or the tube-shaped body is integrally formed from one or more polymer materials and, further, one or more elastic metallic spring bands having shape memory are provided and are configured to deform with the tube-shaped body between the open and closed positions; or the one or more metallic spring bands are embedded within the tube-shaped body; or the one or more metallic spring bands are wrapped around the tube-shaped body; or the tube-shaped body is integrally formed from one or more elastic polymer materials which have shape memory and, further, one or more elastic metallic spring bands having shape memory are provided and configured to deform with the tube-shaped body between the open and closed positions.

More preferably, the tube receiving slot includes a pair of oval shaped receiving channels extending adjacent and parallel to each other through the tube receiving slot and the two ends of the flexible tube are received and aligned one in each of the oval shaped receiving channels. Also, the tube receiving slot has a transverse width and a transverse height and by compressing the tube-shaped body in a direction along the transverse width the tube-shaped body and the receiving slot deform causing the transverse width to decrease and the transverse width to increase thereby placing the tube-shaped body in its open position.

### BRIEF DESCRIPTION OF THE DRAWINGS

The above-mentioned and other features of this invention and the manner of attaining them will become more apparent, and the invention itself will be better understood by reference to the following description of the embodiments of the invention, taken in conjunction with the accompanying drawings, wherein:

FIG. 1 is a perspective view of a nasal cannula constructed in accordance with the principles of the present invention and shown in use hooked over a patient's ears and under the chin and including clamshell-shaped adjustable bolo clasp engaging the flexible tubes of the cannula loop;

FIG. 2 is a perspective view of the nasal cannula shown in FIG. 1;

FIG. 3 is a magnified view of the dash line Circled Detail 3 shown in FIG. 1;

FIGS. 4A-B are perspective views of the adjustable bolo clasp shown in FIG. 1;

FIGS. 4C-D are a top plane view and a side elevation view of the adjustable bolo clasp shown in FIG. 1;

FIG. 5A is a front elevation view of the adjustable bolo clasp of FIG. 1 in the resting position;

FIG. 5B is a cross-section view of the adjustable bolo clasp taken along line 5B-5B shown in FIG. 5A;

FIG. 6A is a front elevation view of the adjustable bolo clasp of FIG. 1 in the clamping position;

FIG. 6B is a cross-section view of the adjustable bolo clasp taken along line 6B-6B shown in FIG. 6A;

FIG. 7A is a front elevation view of the adjustable bolo clasp of FIG. 1 in the open position;

FIG. 7B is a cross-section view of the adjustable bolo clasp taken along line 7B-7B shown in FIG. 7A;

FIG. 8 is a perspective view of the adjustable bolo clasp of FIG. 1 in the open position;

FIG. 9 is a perspective view illustrating the adjustable bolo clasp of FIG. 1 being slidingly adjusted along the flexible tubes;

FIG. 10A is a side plane view of an adjustable bolo clasp similar to the clasp of FIG. 1, in the resting position, and including embedded metallic spring members;

FIG. 10B is a side plane view of the adjustable bolo clasp shown in FIG. 10A in the open position;

FIG. 11A is a perspective view of an adjustable bolo clasp similar to the clasp of FIG. 1, in the resting position, and having metallic arc-shaped spring hinges between and connecting the jaw members;

FIG. 11B is a rear plane view of the adjustable bolo clasp shown in FIG. 11A;

FIG. 11C is a side plane view of the adjustable bolo clasp shown in FIG. 11A;

FIG. 11D is a side plan view of the adjustable bolo clasp shown in FIGS. 11A-B in the open position;

FIG. 12 is a perspective view of another embodiment of a nasal cannula constructed in accordance with the principles of the present invention and shown in use hooked over a patient's ears and under the chin and including a tube-shaped adjustable bolo clasp engaging the flexible tubes of the cannula loop;

FIG. 13 is a magnified view of dash line Circled Detail 13 shown in FIG. 12;

FIG. 14A is a perspective view of the adjustable bolo clasp shown in FIGS. 12 and 13 in the resting position;

FIG. 14B is a front elevation view of adjustable bolo clasp shown in FIG. 14A;

FIG. 14C is a cross-section view of the adjustable bolo clasp taken along line 14C-14C shown in FIG. 14B;

FIG. 15A is a perspective view of the adjustable bolo clasp shown in FIGS. 12 and 13 in the clamping position;

FIG. 15B is a front elevation view of adjustable bolo clasp shown in FIG. 15A;

FIG. 15C is a cross-section view of the adjustable bolo clasp taken along line 15C-15C shown in FIG. 15B;

FIG. 16A is a perspective view of the adjustable bolo clasp shown in FIGS. 12 and 13 in the open position;

FIG. 16B is a front elevation view of adjustable bolo clasp shown in FIG. 16A;

FIG. 16C is a cross-section view of the adjustable bolo clasp taken along line 16C-16C shown in FIG. 16B;

FIG. 17 is a perspective view illustrating the adjustable bolo clasp of FIG. 12 being slidingly adjusted along the flexible tubes;

FIG. 18A is a perspective view of a tube-shaped adjustable bolo clasp similar to the clasp of FIG. 12 and including a pair of metallic spring reinforcing bands;

FIG. 18B is a front elevation view of the adjustable bolo clasp shown in FIG. 18A in the open position; and,

FIG. 18C is a cross-section view of the adjustable bolo clasp taken along line 18C-18C shown in FIG. 18B.

Corresponding reference characters indicate corresponding parts throughout several views. Although the exemplification set out herein illustrates certain embodiments of the invention, the embodiments disclosed below are not intended to be exhaustive or to be construed as limiting the scope of the invention to the precise form disclosed.

### DETAILED DESCRIPTION OF THE PREFERRED EMBODIMENTS

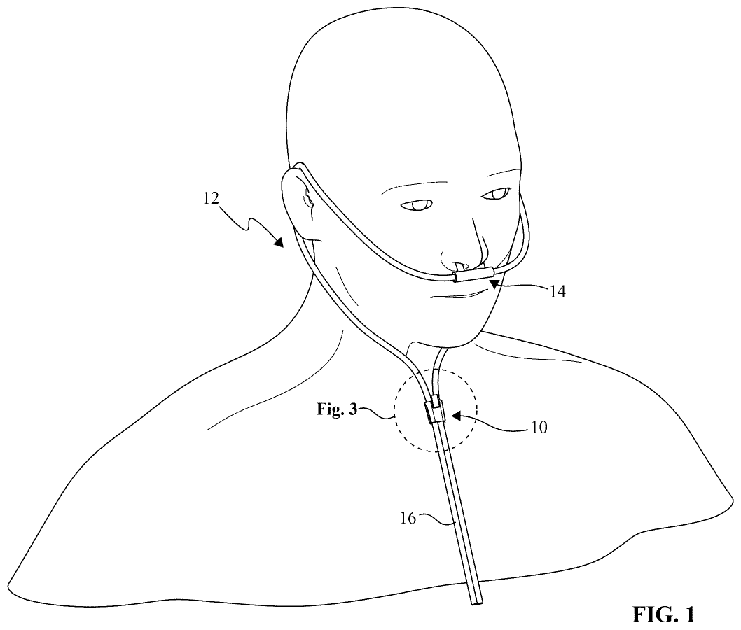

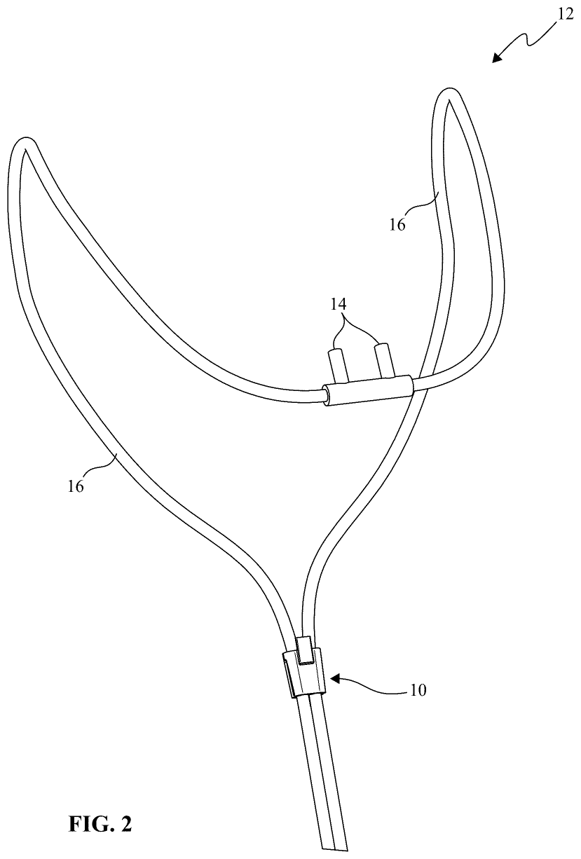

A nasal cannula 12 including an adjustable bolo clasp 10, 100 constructed in accordance with the principles of the present invention is shown in FIGS. 1, 2 and 12. The adjustable bolo clasp 10, 100 is configured to selectively engage and clamp against/be secured to the flexible tubes 16 and is used to selectively adjust the fitment of the nasal cannula 12. More particularly, the nasal cannula 12 comprises a pair of prongs 14, which are fluidly connected via a pair of flexible tubes 16 to an oxygen source in a known and customary manner such as, for example, a portable oxygen tank or oxygen generator (not shown). The nasal cannula 12 is used by inserting the prongs 14 into the patient's nostrils and pumping oxygen from the oxygen source through the flexible tubes 16 and the prongs 14 into the patient nostrils.

As best seen in FIGS. 1, 2 and 12, the flexible tubes 16 form a cannula loop which extends along and between the upper lip and the nose and is hooked over the patient's ears and under the chin to thereby retain the prongs 14 in the nostrils. In use, the adjustable bolo clasp 10, 100 selectively engages and clamps against/is secured to the flexible tubes 16 for closing the bottom of the loop. The adjustable bolo clasp 10 can be slidingly adjusted along the length of the flexible tubes 16 for tightening/decreasing the size of the loop or loosening/expanding the size of loop and thereby adjusting the fitment of the flexible tubes 16 over and around the patient's ears and under the chin. By tightening/decreasing the size of the loop the nasal cannula 12 can be adjusted to prevent inadvertent displacement/removal of the prongs 14 such as while a patient is sleeping and turns their head thereby forcing the nasal cannula against a pillow. By loosening/expanding the size of loop the nasal cannula 12 can more easily be placed on and removed from the patient.

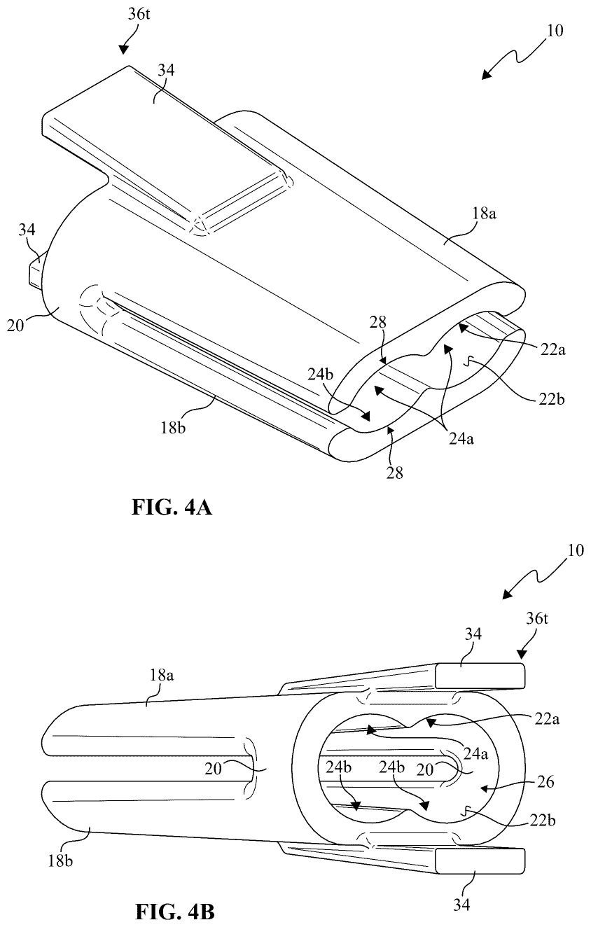

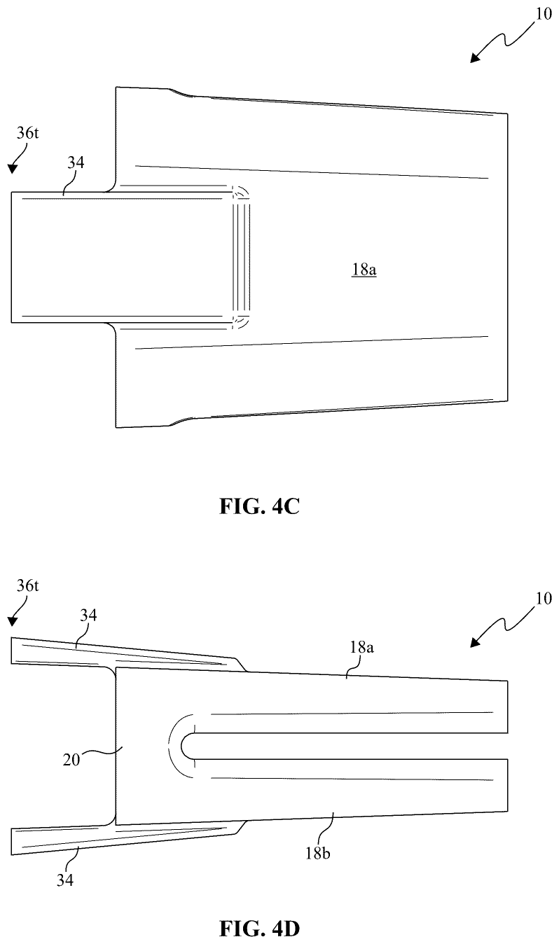

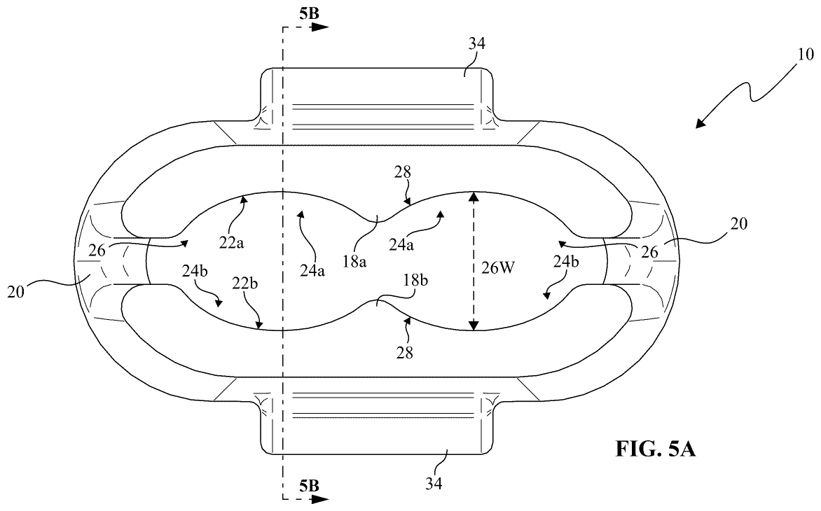

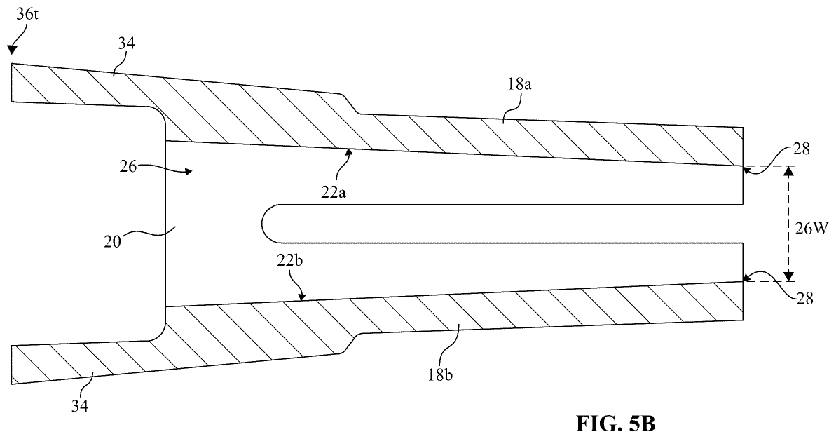

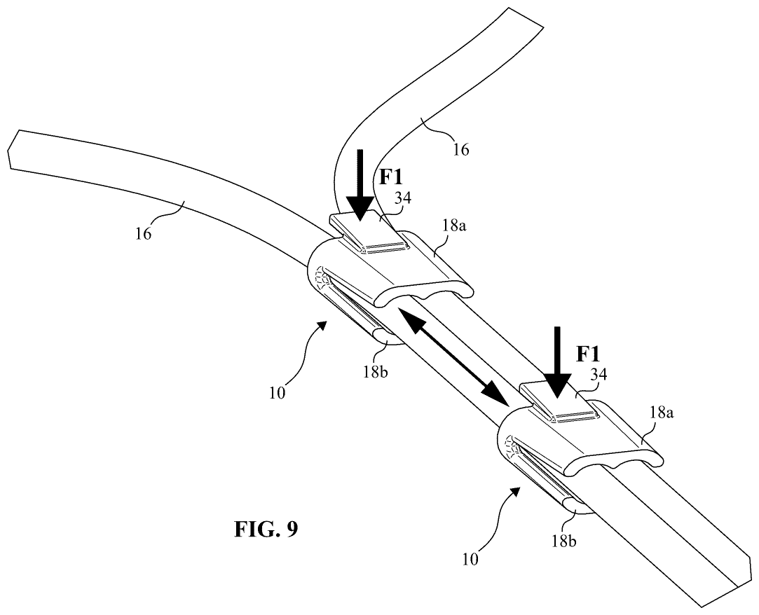

In a first embodiment (FIGS. 1-11), the adjustable bolo clasp 10 can be a clamshell-shaped clasp comprising a pair of elongated jaw members 18a, 18b connected together by a pair of hinges 20. The jaw members 18a, 18b extend generally parallel to and are spaced apart from each other for receiving therebetween the pair of flexible tubes 16 that lead to and form the loop of the cannula 12. As best seen in FIGS. 4A, 5A, and 5B, the jaw members 18a, 18b each include a respective tube engaging surface 22a, 22b. The engaging surface 22a of jaw member 18a faces the opposing engaging surface 22b of jaw member 18b. Preferably, the tube engaging surface 22a is shaped to comprise a pair of longitudinally extending grooves/troughs 24a and the tube engaging surface 22b is shaped to comprise a pair of longitudinally extending grooves/troughs 24b. The grooves 24a of engaging surface 22a are longitudinally aligned with the grooves 24b of engaging surface 22b and thereby define a pair of longitudinally extending oval-shaped tube receiving channels 26 therebetween. Each of the oval-shaped tube receiving channels 26 are configured to receive a flexible tube 16 therethrough and help to align and retain the pair of flexible tubes 16 within and parallel to each other within bolo clasp 10 during and after the clasp is slidingly longitudinally adjusted along the tubes.

Preferably, the bolo clasp 10 is configured such that the leading areas and edges 28 of the tube engaging surfaces 22a, 22b can selectively engage and clamp against/therebetween the flexible tubes 16 for increasing the friction between the jaw members 18a, 18b and the flexible tubes 16 and thereby prevent slippage therebetween. Specifically, clamping the flexible tubes 16 against/between the leading areas/edges 28 causes the leading areas/edges 28 to impinge or “bite” into the flexible tubes 16 which thereby prevents the adjustable bolo clasp 10 from sliding longitudinally along the flexible tubes 16.

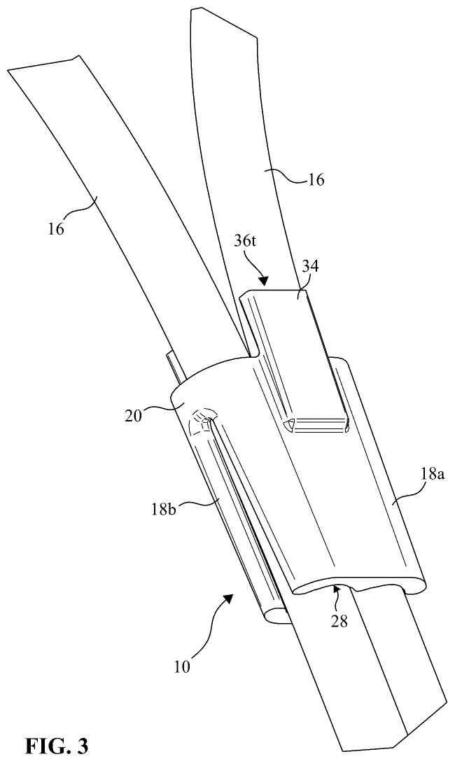

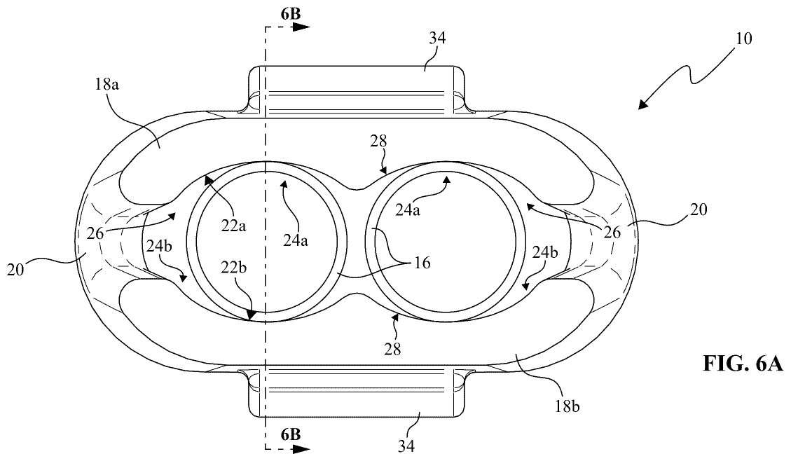

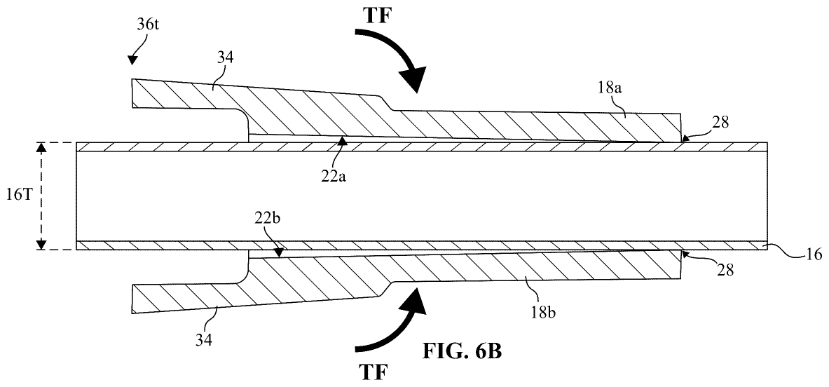

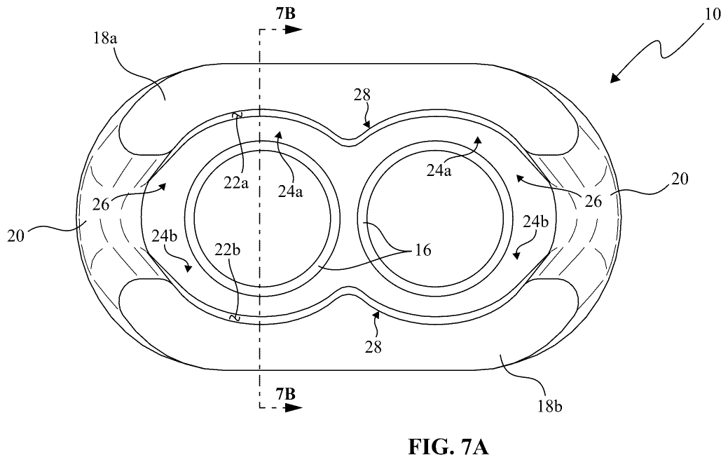

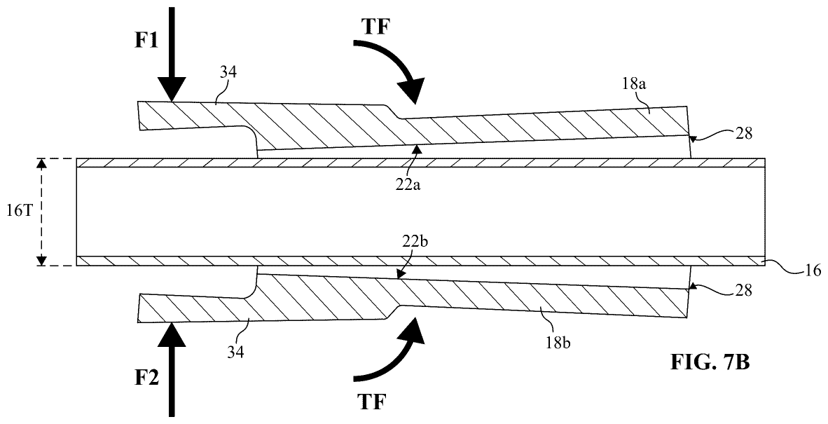

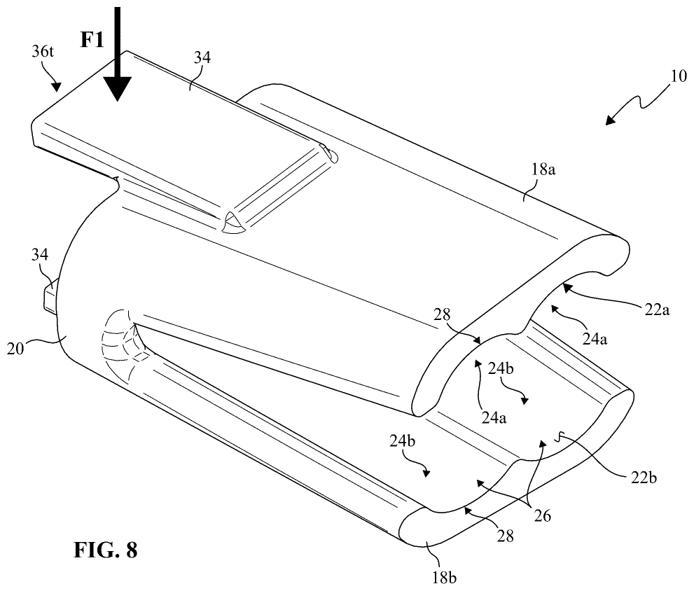

The bolo clasp 10 is preferably made of a polymer material such as polyurethane, polyvinyl chloride, silicon rubber, styrene-butadiene rubber and/or other similar materials such as by injection molding and, as best seen in FIGS. 4B, 4D, 5A, and 5B, the hinges 20 can be wall-shaped sections that are integrally formed with and extend between the jaw members 18a, 18b, one on either side of the adjustable bolo clasp 10. The polymer or other similar material of the wall-shaped sections/hinges 20 is flexible for thereby forming an integral or “live hinge” but also has “shape memory” whereby, as the jaw members 18a, 18b pivot thereabout to an open position, the wall-shaped sections/hinges 20 deform and are subjected to compressive and tensile/expansive forces against the deformation force of the shape memory and thereby store elastic potential energy (FIGS. 7A-B, 8 and 9) and, when the jaw members 18a, 18b are in a resting position whereat the wall-shaped sections/hinges 20 are not deformed from their original molded shapes, the wall-shaped sections/hinges 20 do not comprise elastic potential energy (FIGS. 4A-D and 5A-B). That is, the live hinges 20 are configured to act like spring hinges that allow the jaw members 18a, 18b to be selectively pivoted thereabout against the deformation/spring force away from each other for disengaging from the flexible tubes 16, as shown in FIGS. 7B and 8, and that can also forcibly elastically retract the jaw members 18a, 18b towards each other for clamping the flexible tubes 16 against/between the jaw members 18a, 18b, as shown in FIGS. 3 and 6B.

The wall-shaped sections/live hinges 20 also function as fulcrum points. That is, the jaw members 18a, 18b preferably each include an integrally formed lever tab 34 which extends towards a trailing end 36t of the adjustable bolo clasp 10. The lever tabs 34 can be used for pivoting the jaw members 18a, 18b between the resting, clamping, and open positions. For example, as illustrated in FIGS. 5B, 6B, and 7B, applying the opposing forces F1, F2, such as by depressing the lever tabs 34 towards each other between one's thumb and finger, causes the jaw members 18a, 18b to pivot about the wall-shaped sections/live hinges and fulcrum points 20 to the open position for allowing the flexible tubes 16 to slide through and between the jaw members 18a, 18b.

As should now be appreciated, the adjustable bolo clasp 10 is configured such that the jaw members 18a, 18b can be selectively pivoted between a resting position (FIGS. 4A-D and 5A-B), a clamping position (FIGS. 3 and 6A-B), and an open position (FIGS. 7A-B, 8, and 9). The resting position is the position wherein opposing forces F1, F2 are not being applied to the lever tabs 34 and the jaw members 18a, 18b are not obstructed such as by the tubes 16 and, as also described hereinabove, the position/shape in which the jaw members 18a, 18b were originally molded/formed (FIGS. 4A-D and 5A-B). The resting position is also the position in which the wall-shaped sections/live hinges 20 do not comprise elastic potential energy. The clamping position is the position wherein opposing forces F1, F2 are not being applied to the lever tabs 34 and wherein the jaw members 18a, 18b are pivoted about hinge 20 slightly apart from each other because they are being obstructed by the flexible tubes 16 and the leading areas/edges 28 thereof are being clamped onto/being forced against the flexible tubes 16 to prevent sliding of the bolo clasp 10 along the flexible tubes 16 (FIGS. 3 and 6A-B). In the clamping position, the wall-shaped sections/live hinges 20 comprise elastic potential energy which forces the jaw members 18a, 18b towards/against the flexible tubes 16. The open position is the position wherein the opposing forces F1, F2 have been applied to the lever tabs 34 and the jaw members 18a, 18b are pivoted about the wall-shaped sections/live hinges and fulcrum points 20 apart from each other and the leading areas/edges 28 thereof are disengaged from the flexible tubes 16 such that the bolo clasp 10 can be slidingly adjusted along the flexible tubes 16 (FIGS. 7A-B, 8, and 9). The open position is also the position in which the wall-shaped sections/live hinges 20 comprise generally maximum elastic potential energy.

As mentioned hereinabove, because the hinges 20 are made of a polymer or other similar material and have “shape memory”, as the jaw members 18a, 18b are pivoted away from each other about the hinges 20, the hinges 20 elastically deform and are placed under compression and tension or, stated differently, experience deformation forces resulting in stored elastic potential energy. More particularly, as the jaw members 18a, 18b pivot away from each other, the live hinges 20 deform and stretch whereby a spring tension force TF is generated within the live hinges 20. This spring tension force TF is illustratively shown in FIGS. 6B and 7B as a force vector which is generated by the deformation of the material and which is applied to the jaw members 18a, 18b opposing the pivoting about the hinges/fulcrum points 20 caused by the forces F1, F2. When the opposing forces F1, F2 are released, the tension force TF causes the live hinges 20 to retract towards their original shape (i.e., the shape of the live hinges 20 in the resting position) which retracts the jaw members 18a, 18b towards each other and clamps/forces the leading areas/edges 28 of the jaw members 18a, 18b against the flexible tubes 16. Hence, the live hinges/fulcrum points 20 act like spring hinges that allow the jaw members 18a, 18b to pivot thereabout and also forcibly retract the jaw members 18a, 18b together for clamping the flexible tubes 16 between the leading areas/edges 28 thereof.

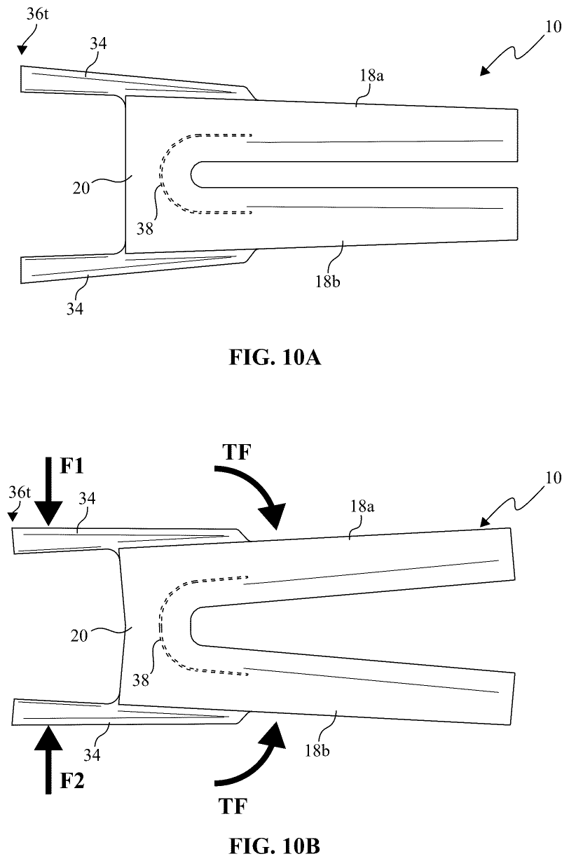

Turning now to FIGS. 10A-B, the adjustable bolo clasp 10 can further include one or more springs members 38. The spring members 38 can be, for example, arc-shaped springs which are formed from a flexible spring material such as, for example, spring steel, stainless steel, phosphor bronze, or other metallic materials commonly used for springs, and can be cast or embedded into the live hinges 20 and the jaw members 18a, 18b. In use, the spring members 38 flex and elastically deform along with the live hinges 20 for increasing the tension force TF generated thereby and also for reinforcing and helping the live hinges 20 to retract and return to their original shape when the opposing forces F1, F2 are released. More particularly, when the opposing forces F1, F2 are applied for pivoting the jaw members 18a, 18b to the open position, the spring members 38 flex and elastically deform along with the live hinges 20. As mentioned above, this elastic deformation generates a tension force TF which opposes the pivoting of the jaw members 18a, 18b apart from each other. The spring members 38 are configured such that the elastic deformation of both the spring members 38 and the live hinges 20 generates a greater tension force TF than the tension force TF generated by the elastic deformation of the live hinges 20 alone. When the opposing forces F1, F2 are released, the spring members 38 and the polymer or similar material live hinges 20 retract toward their original shape thereby together forcibly retracting the jaw members 18a, 18b together for clamping the flexible tubes 16 between the leading areas/edges 28 thereof.

Alternatively, the bolo clasp 10 can be injection molded and made with a polymer or other similar material which does not have sufficient “shape memory” to generate a deformation force/elastic potential energy in the live hinges 20 thereof, but wherein a spring member 38 is cast or embedded into the live hinges 20 and the jaw members 18a, 18b as described hereinabove and shown in FIGS. 10A-B. The spring members 38 in this embodiment are configured such that they primarily or alone generate the tension force TF so that, when the opposing forces F1, F2 are released, the spring members 38 retract and return to their original shape which thereby forcibly retracts the jaw members 18a, 18b together for clamping the flexible tubes 16 between the leading areas/edges 28 thereof.

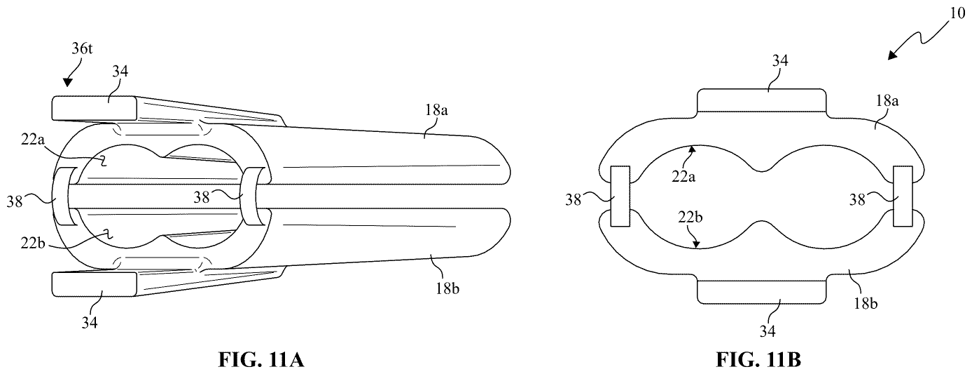

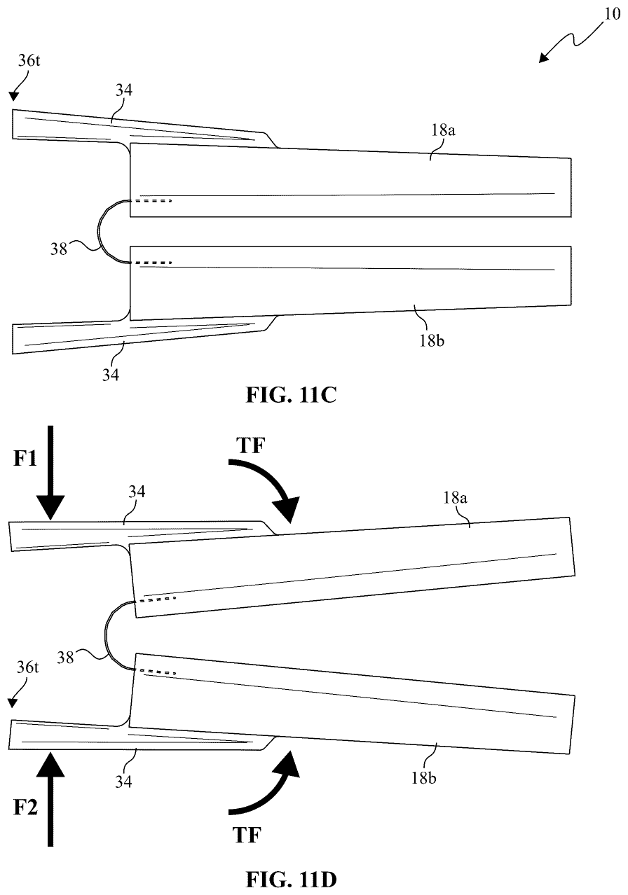

As shown in FIGS. 11A-D, the spring members 38 can also be used to replace the live hinges 20 entirely. In this embodiment, the spring members 38 are secured to the jaw members 18a, 18b by for example casting or embedding the spring members 38 into the jaw members 18a, 18b during manufacture thereof. In operation, the arc-shaped springs 38 function similar to the integrally formed live hinges/fulcrum points 20 wherein, as the jaw members 18a, 18b pivot away from each other, the springs 38 elastically deform and generate a spring tension force TF. When the opposing forces F1, F2 are released, the springs 38 retract and return towards their original shape which forcibly retracts the jaw members 18a, 18b towards each other and clamps the leading areas/edges 28 of the jaw members 18a, 18b against the flexible tubes 16.

Preferably, as best seen in FIGS. 5A-B, 6B and 7B, the bolo clasp 10 is configured such that, when the jaw members 18a, 18b are in the resting position, the width 26W between the leading areas/edges 28 of the oval-shaped tube receiving channels 26 is slightly narrower/smaller than the thickness/outside diameter 16T of the flexible tubes 16. Also, the width of the oval-shaped tube receiving channels 26 in the area adjacent to/between the live hinges/fulcrum points 20 is slightly wider/larger than the thickness/outside diameter 16T of the flexible tubes 16. Accordingly, for inserting the flexible tubes 16 between the jaw members 18a, 18b and within the channels 26 and for disengaging from the flexible tubes 16 and slidingly adjusting the bolo clasp 10 along the tubes, opposing forces F1, F2 are applied to the lever tabs 34 for thereby increasing the width 26W at the leading areas/edges 28 to a distance also greater than the thickness/outside diameter 16T of the flexible tubes 16.

After the flexible tubes 16 are inserted through or are otherwise placed in position extending through the bolo clasp 10 and between the jaw members 18a, 18b and the nasal cannula 12 is thereby ready for use, the bolo clasp 10 can be disengaged from the flexible tubes 16, such as by depressing the lever tabs 34 towards each other between one's thumb and finger thereby pivoting the jaw members 18a, 18b to their open position as described hereinabove, and slidingly adjusting it along the flexible tubes 16 to loosen/expand the size of loop. The nasal cannula 12 can then be placed on the patient as described hereinabove. The nasal cannula 12 can then be tightened/better fitted on the patient by similarly disengaging the bolo clasp 10 from the flexible tubes 16 and slidingly adjusting it along the tubes for thereby decreasing the size of the loop. Of course, while the nasal cannula 12 is on the patient, the bolo clasp 10 is in its clamping position so that the bolo clasp 10 is prevented from sliding along the flexible tubes 16, thereby securely maintaining the nasal cannula 12 on the patient and preventing inadvertent displacement/removal of the prongs 14 thereof from the nostrils. For removing the nasal cannula 12 from the patient, the bolo clasp 10 can similarly be disengaged from the flexible tubes 16 and slidingly adjusted for thereby increasing the size of the loop and hence easily removing the nasal cannula 12 from the patient.

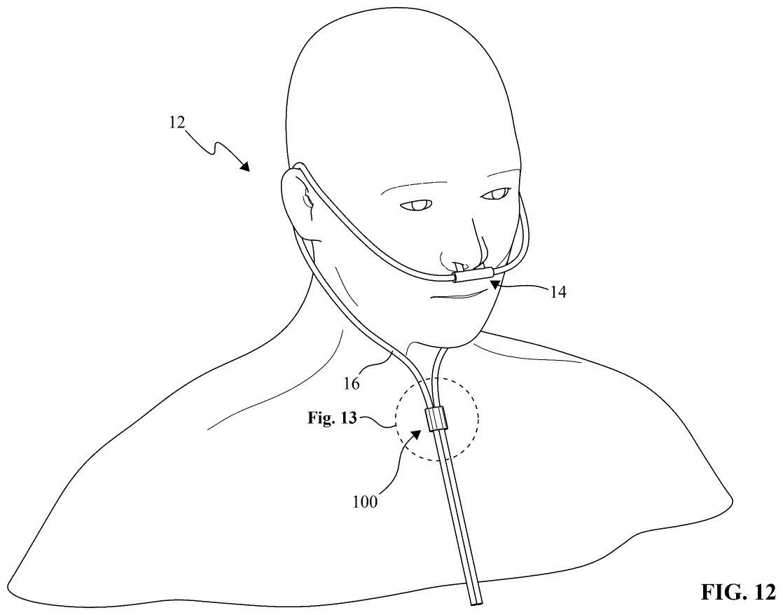

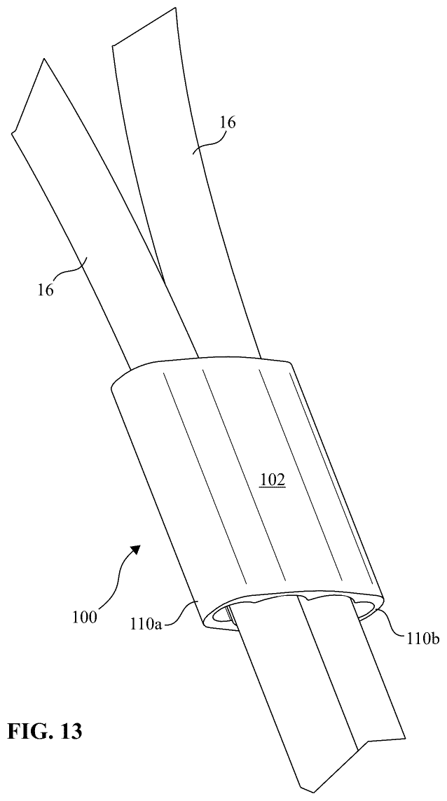

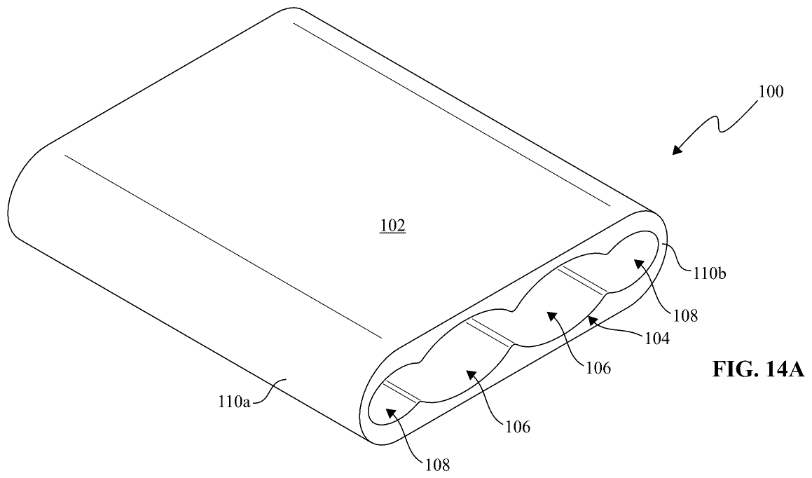

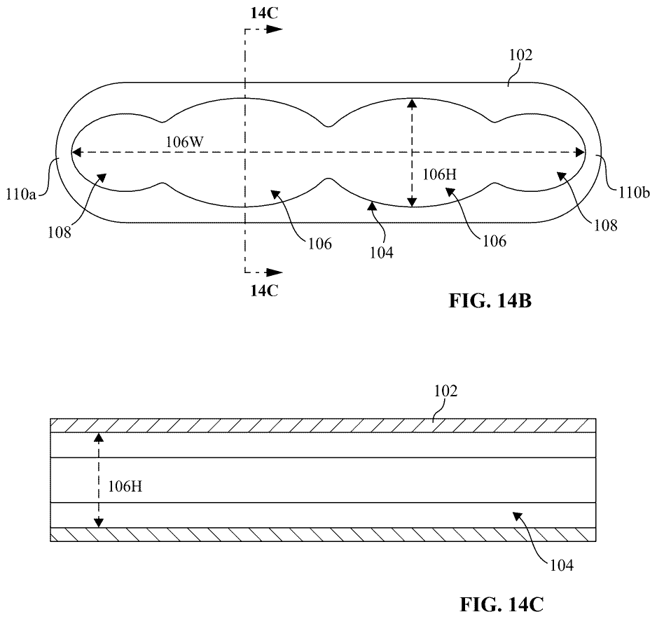

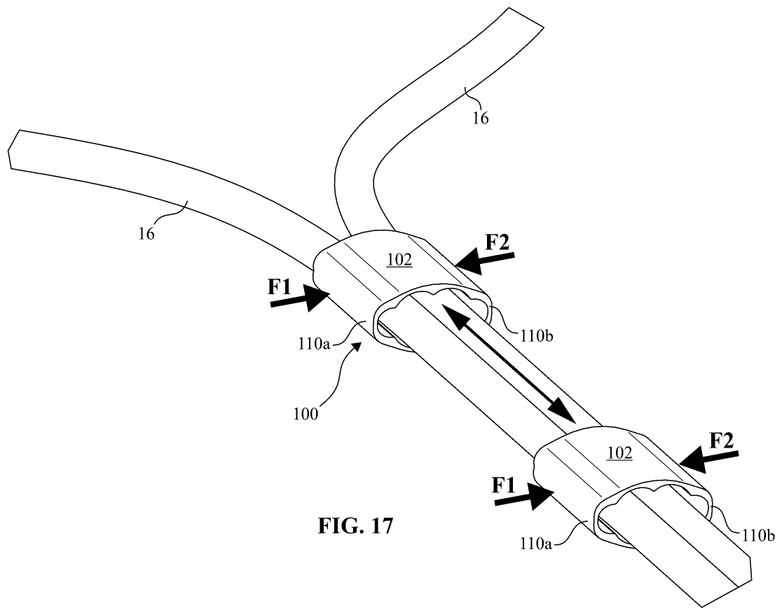

Turning to now FIGS. 12-18, in another embodiment of the nasal cannula 12, the adjustable bolo clasp 100 can comprise a flexible tube-shaped body 102 having a tube receiving slot 104 extending longitudinally therethrough. The body/wall 102 of the clasp is preferably integrally formed such as by injection molding or otherwise formed from a flexible, elastic material, such as, for example, a rubber material, a flexible polyurethane material, polyvinyl chloride, a silicon rubber material, a styrene-butadiene rubber material, and/or other flexible, elastic polymer materials or, alternatively, can be formed and made of for example, spring steel, stainless steel, phosphor bronze, or other metallic materials commonly used for manufacturing springs, and can be shaped, for example, like a flattened cylinder (see e.g., FIGS. 14A and 14B). The tube receiving slot 104 extends longitudinally through the body 102 and, as best seen in FIG. 14B, preferably has a perimeter profile shaped like four overlapping ovals. Put another way, the slot 104 preferably comprises a pair of oval-shaped tube receiving channels 106 which extend adjacent and parallel to each other and a pair of oval-shaped relief slots 108 which extend one on either side of the pair of tube receiving channels 106. The tube receiving channels 106 are configured to receive and align the flexible tubes 16 longitudinally within the bolo clasp 100. The relief slots 108 create space on either side of the tube receiving channels 106 such that the body 102 can be compressed in the direction along its transverse width 106W without the longitudinal sides 110a, 110b of the body engaging the flexible tubes 16.

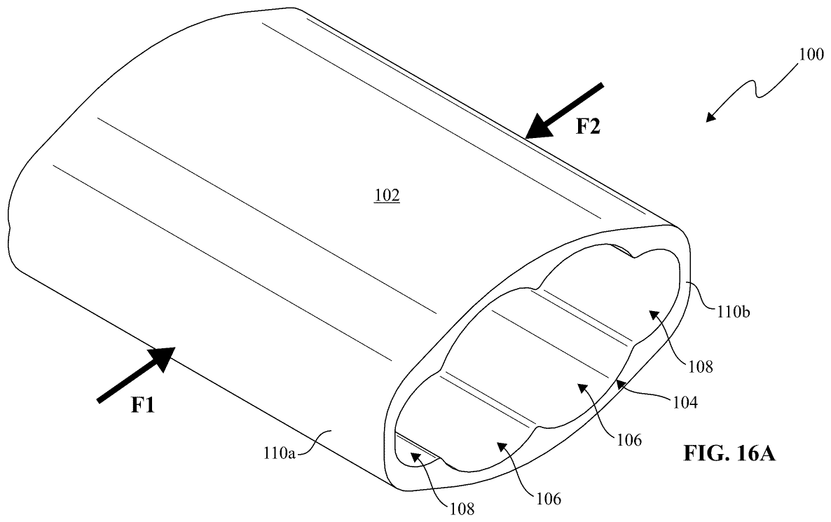

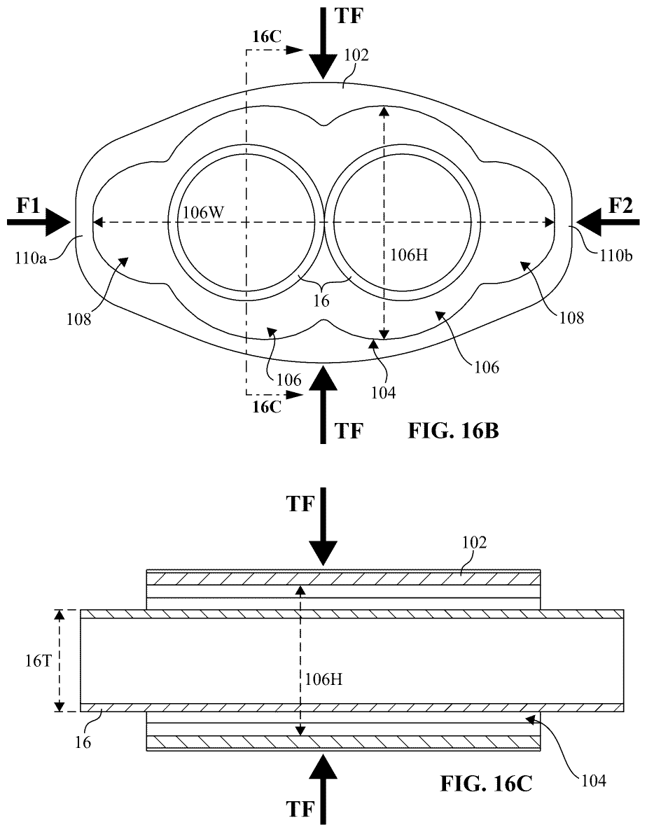

The bolo clasp 100 is used by selectively applying opposing forces F1, F2 to the longitudinal sides 110a, 110b of the body 102, such as by depressing the bolo clasp sides 110a, 110b/compressing the bolo clasp at its sides 110a, 110b between one's thumb and finger. These opposing forces F1, F2 cause the body 102 to elastically deform towards a more circular shaped cylinder for expanding the tube receiving channels 106 and placing the bolo clasp 100 in an open position as shown in FIGS. 16A-C and thereby allowing the bolo clasp 100 to be slidingly adjusted along the flexible tubes 16. The material of the tube-shaped body 102 is flexible for thereby elastically deforming but also has “shape memory” whereby, as the bolo clasp 100 is placed in its open position, the tube shaped body/wall 102 elastically deforms against the deformation force of the shape memory and is subjected to compressive and tensile/expansive forces for thereby storing elastic potential energy (FIGS. 16A-C) and, when the bolo clasp 100 is in a resting position whereat the body 102 is not deformed from its original molded/formed shape (FIGS. 14A-C), the body 102 does not comprise elastic potential energy.

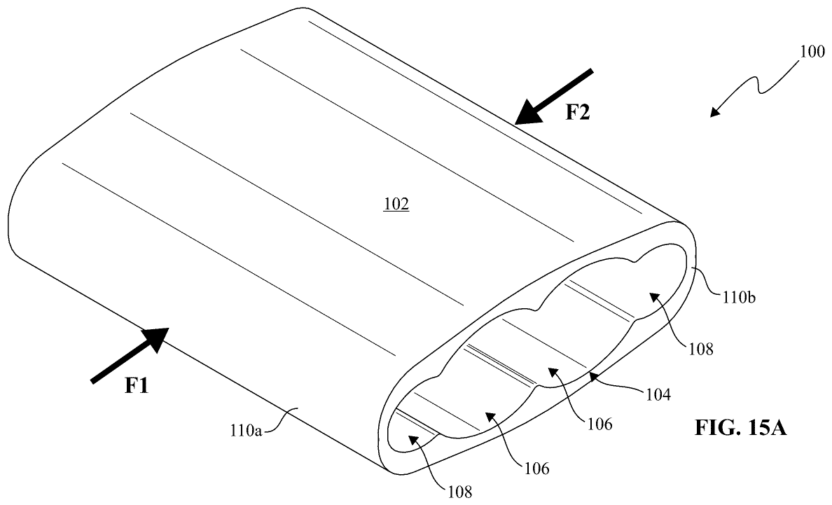

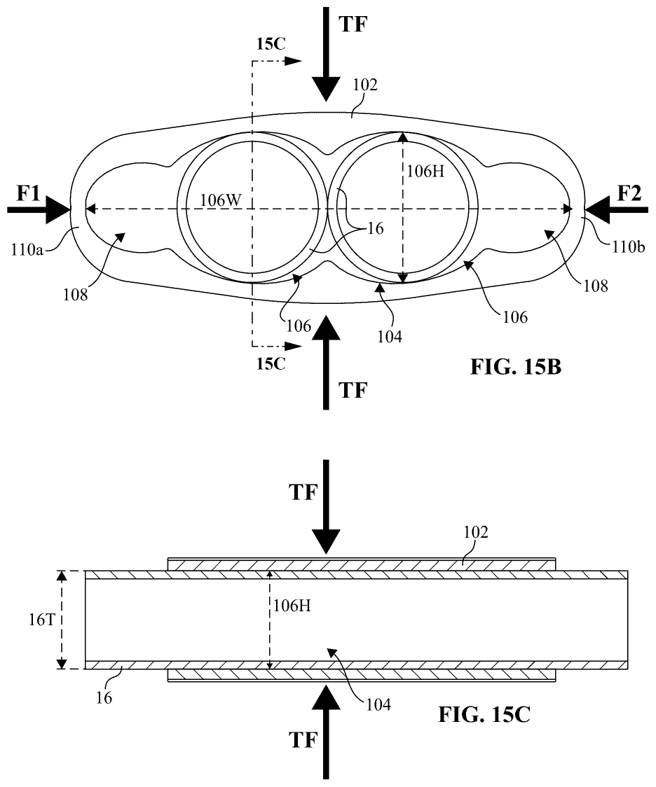

More particularly, the bolo clasp 100 is configured to expand and retract between a resting position (FIGS. 14A-C), a clamping position (FIGS. 15A-C), and an open position (FIG. 16A-C). The resting position is the position in which the body/wall 102 was originally molded/formed and which the bolo clasp 100 naturally returns to when opposing forces F1, F2 are not being applied to the longitudinal sides 110a, 110b and there are no obstructions, such as tubes 16, in the tube receiving slot 104 (FIGS. 14A-C). The resting position is also the position in which the body/wall 102 does not comprise elastic potential energy, and the resting transverse height 106H and the resting transverse width 106W of receiving channels 106 are at their minimum as shown in FIGS. 14A-C. The resting transverse height/distance 106W as shown in FIGS. 14A-C is also smaller than the thickness/outside diameter 16T of the flexible tubes 16. The clamping position is the position wherein opposing forces F1, F2 are not being applied to the longitudinal sides 110a, 110b and wherein the body/wall 102 is slightly deformed and the transverse height 106H of the receiving channels 106 is, as shown in FIGS. 15A-C, slightly greater than the resting transverse height 106H of FIGS. 14A-C because the body/wall 102 is being obstructed by the flexible tubes 16 positioned within the oval-shaped tube receiving channels 106. The clamping position is essentially the position in which the body/wall 102 is clamped onto/cinched around the flexible tubes 16 and prevents sliding of the bolo clasp 100 along the flexible tubes 16 (FIGS. 15A-C). In the clamping position, the body/wall 102 comprises elastic potential energy which forces it towards/against the flexible tubes 16. The open position is the position wherein the opposing forces F1, F2 have been applied to the longitudinal sides 110a, 110b and wherein the body/wall 102 is further elastically deformed and the transverse height 106H of the receiving channels 106 is, as shown in FIGS. 16A-C, expanded to a height/distance greater than the thickness/outside diameter 16T of the flexible tubes 16 such that the tubes 16 can freely slide therethrough and bolo clasp 100 can be slidingly adjusted along the flexible tubes. The open position is also the position in which the body/wall 102 comprises generally maximum elastic potential energy.

As illustrated in FIGS. 14-17, when the opposing forces F1, F2 are applied to the body longitudinal sides 110a, 110b such as between one's thumb and finger, the body 102 elastically compresses/deforms in the direction of the channel transverse width 106W which causes the body 102 to elastically expand/deform in the direction of the channel transverse height 106H thereby placing the bolo clasp in its open position and, as mentioned hereinabove, results in elastic potential energy being stored in the body/wall 102. The stored elastic potential energy essentially creates a spring tension force TF which is illustratively shown in FIGS. 15B-C, 16B-C and 18B-C. When the opposing forces F1, F2 are released, the tension forces TF cause the body/wall 102 to retract towards its originally molded/resting position thereby forcing the body/wall 102 to clamp onto/be cinched around the flexible tubes 16 as illustrated by the clamping position shown in FIGS. 15A-C, thereby preventing sliding of the bolo clasp 100 along the flexible tubes.

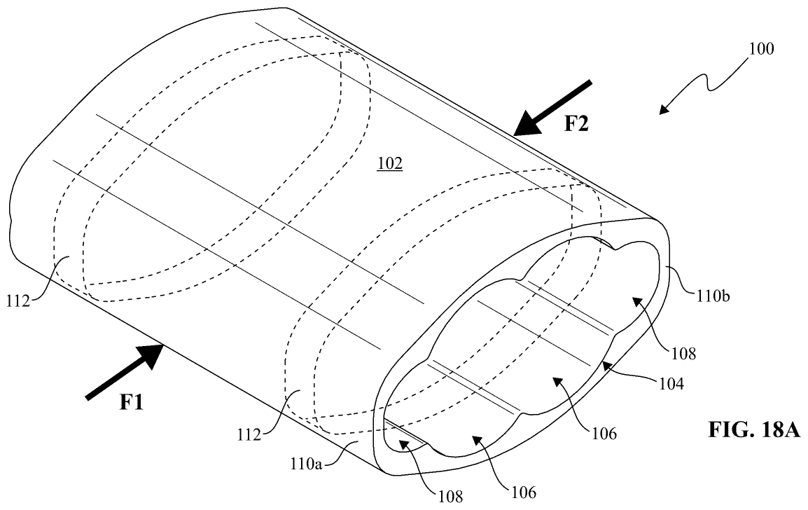

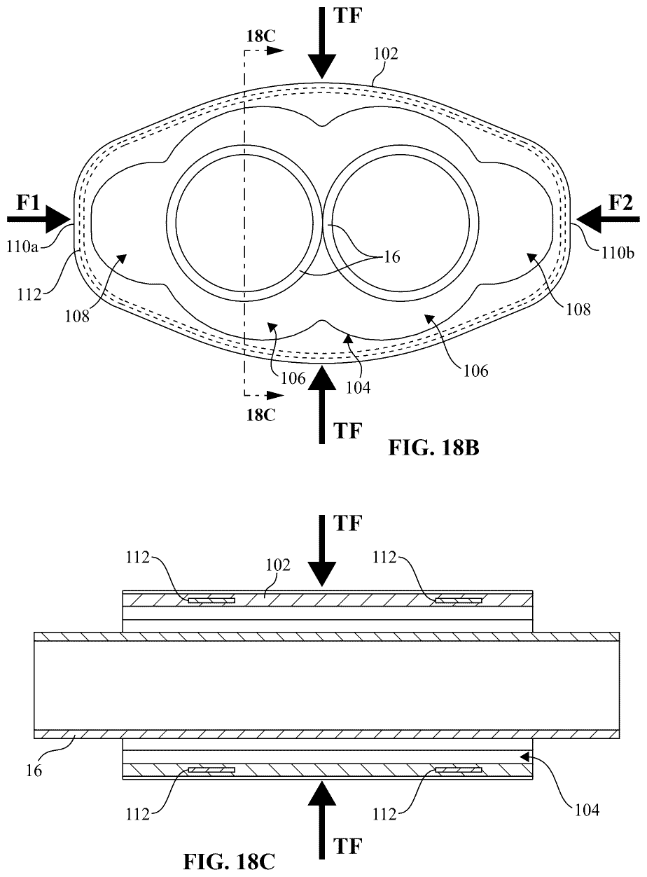

As shown in FIGS. 18A-C, when the body wall 102 is made of elastic polymer materials, the bolo clasp 100 can also include one or more metallic spring reinforcing bands 112 which are preferably embedded into the body/wall 102 or which can be provided outside of and wrapped around the body/wall 102 and configured to elastically deform together with the body 102. The reinforcing bands 112 can be formed from, for example, spring steel, stainless steel, phosphor bronze, or other metallic materials commonly used for manufacturing springs and reinforce the body 102. The bands 112 have “shape memory” whereby, as the bolo clasp 100 is placed in its open position by applying forces F1, F2 to the body longitudinal sides 110a, 110b, the bands 112 elastically deform against the deformation force of their shape memory and are subjected to compressive and tensile/expansive forces for thereby storing elastic potential energy and, when the bolo clasp 100 is in a resting position whereat the body 102 is not deformed from its original molded shape as for example shown in FIGS. 14A-C, the bands 112 do not comprise elastic potential energy. Hence, the stored elastic potential energy of the bands 112 essentially creates a spring tension force TF which is illustratively shown in FIGS. 18B-C so that, when the opposing forces F1, F2 are released, the tension forces TF cause the bands 112 and the body/wall 102 to retract towards their originally molded/resting position thereby forcing the body/wall 102 to clamp onto/be cinched around the flexible tubes 16 as illustrated by the clamping position shown, for example, in FIGS. 15A-C, thereby preventing sliding of the bolo clasp 100 along the flexible tubes. In this regard, if the body/wall 102 also has “shape memory”, the elastic deformation of both the body/wall 102 and bands 112 both generate elastic potential energy and a tension TF whereas, if the body/wall 102 does not have sufficient “shape memory” to generate a deformation force/elastic potential energy, the bands 112 are configured such that they primarily or alone generate the tension force TF required to forcibly clamp the body/wall 102 onto the flexible tubes 16 and thereby prevent sliding of the bolo clasp 100 along the tubes.

The bolo clasp 100 is used similar to the bolo clasp 10. That is, after the flexible tubes 16 are inserted through or are otherwise placed in position extending through the receiving channels 106 and the nasal cannula 12 is thereby ready for use, the bolo clasp 100 can be disengaged from the flexible tubes 16, such as by depressing the bolo clasp sides 110a, 110b between one's thumb and finger and thereby deforming and placing the body/wall 102 in its open position as described hereinabove, and slidingly adjusting it along the flexible tubes 16 to loosen/expand the size of loop. The nasal cannula 12 can then be placed on the patient as described hereinabove. The nasal cannula 12 can then be tightened/better fitted on the patient by similarly disengaging the bolo clasp 100 from the flexible tubes 16 and slidingly adjusting it along the tubes for thereby decreasing the size of the loop. While the nasal cannula 12 is on the patient, the bolo clasp 100 is in its clamping position so that the bolo clasp 100 is prevented from sliding along the flexible tubes 16, thereby securely maintaining the nasal cannula 12 on the patient and preventing inadvertent displacement/removal of the prongs 14 thereof from the nostrils. For removing the nasal cannula 12 from the patient, the bolo clasp 100 can similarly be disengaged from the flexible tubes 16 and slidingly adjusted for thereby increasing the size of the loop and hence easily removing the nasal cannula 12 from the patient.

While this invention has been described as having an exemplary design, the present invention may be further modified within the spirit and scope of this disclosure. This application is therefore intended to cover any variations, uses, or adaptations of the invention using its general principles.

1. A nasal cannula comprising:
a flexible tube fluidly connected to a pair of prongs adapted to be received in nose nostrils for delivering oxygen to the prongs;
a bolo clasp comprising a pair of jaw members pivotally secured together with one or more hinges and defining one or more spaces therebetween;
wherein two ends of the flexible tube are received through the bolo clasp one or more spaces whereby a cannula loop is defined by the flexible tube and is closed by the bolo clasp; and,
wherein the jaw members pivot about the one or more hinges between an open position whereat the bolo clasp can be slidingly adjusted along the flexible tubes for thereby adjusting the size of the loop and a clamping position whereat the jaw members engage the flexible tubes and prevent sliding of the bolo clasp along the flexible tubes.

2. The nasal cannula of claim 1 wherein the one or more hinges are spring hinges.

3. The nasal cannula of claim 1 wherein the jaw members and the one or more hinges are integrally formed from one or more polymer materials and the one or more hinges are live hinges.

4. The nasal cannula of claim 1 wherein the jaw members and the one or more hinges are integrally formed from one or more polymer materials and the one or more hinges are elastic live hinges comprising shape memory.

5. The nasal cannula of claim 1 wherein the jaw members and one or more hinges are integrally formed from one or more polymer materials and the one or more hinges are live hinges, and further comprising one or more metallic spring members embedded within each of the one or more live hinges.

6. The nasal cannula of claim 1 wherein the jaw members and the one or more hinges are integrally formed from one or more polymer materials and the one or more hinges are elastic live hinges comprising shape memory, and further comprising one or more metallic spring members embedded within each of the one or more live hinges.

7. The nasal cannula of claim 1 wherein the one or more hinges comprise a metallic spring member.

8. The nasal cannula of claim 1 wherein each jaw member includes a lever tab and the one or more hinges are located between the jaw members and lever tabs and the hinges also function as fulcrum points.

9. The nasal cannula of claim 8 wherein the one or more hinges are spring hinges.

10. The nasal cannula of claim 8 wherein the jaw members and the one or more hinges are integrally formed from one or more polymer materials and the one or more hinges are live hinges.

11. The nasal cannula of claim 8 wherein the jaw members and the one or more hinges are integrally formed from one or more polymer materials and the one or more hinges are elastic live hinges comprising shape memory.

12. The nasal cannula of claim 8 wherein the jaw members and one or more hinges are integrally formed from one or more polymer materials and the one or more hinges are live hinges, and further comprising one or more metallic spring members embedded within each of the one or more live hinges.

13. The nasal cannula of claim 8 wherein the jaw members and the one or more hinges are integrally formed from one or more polymer materials and the one or more hinges are elastic live hinges comprising shape memory, and further comprising one or more metallic spring members embedded within each of the one or more live hinges.

14. The nasal cannula of claim 8 wherein the one or more hinges comprise a metallic spring member.

15. The nasal cannula of claim 8 wherein each jaw member comprises an impingement area adapted to engage the flexible tubes and prevent sliding of the bolo clasp along the flexible tubes.

16. The nasal cannula of claim 8 wherein each jaw member comprises a pair of grooves facing the one or more spaces between the jaws and aligned with the pair of grooves of the opposing jaw thereby defining a pair of tube receiving channels whereat the flexible tubes are received.

17. The nasal cannula of claim 8 wherein the polymer material is one or more of polyurethane, polyvinyl chloride, silicon rubber and styrene-butadiene rubber.

18. The nasal cannula of claim 1 wherein each jaw member comprises an impingement area adapted to engage the flexible tubes and prevent sliding of the bolo clasp along the flexible tubes.

19. The nasal cannula of claim 1 wherein each jaw member comprises a pair of grooves facing the one or more spaces between the jaws and aligned with the pair of grooves of the opposing jaw thereby defining a pair of tube receiving channels whereat the flexible tubes are received.

20. The nasal cannula of claim 1 wherein the polymer material is one or more of polyurethane, polyvinyl chloride, silicon rubber and styrene-butadiene rubber.

21-42. (canceled)
